(19) United States
(12) Patent Application Publication
(10) Pub. No.: US 2025/0288768 A1
(43) Pub. Date: Sep. 18, 2025

(54) PEEP VALVE APPARATUS

(71) Applicant: Engineered Medical Systems, Inc., Indianapolis, IN (US)

(72) Inventor: Scott Weingart, Setauke-East Setauket, NY (US)

(73) Assignee: Engineered Medical Systems, Inc., Indianapolis, IN (US)

(21) Appl. No.: 18/605,181

(22) Filed: Mar. 14, 2024

(51) Int. Cl. A61M 16/08; A61M 16/04; A61M 39/26

(52) U.S. Cl. CPC A61M 16/0833; A61M 16/0463; A61M 39/26; A61M 2205/02

(57) ABSTRACT

An apparatus for use with an endotracheal tube including a body defining a cavity therein. The body has a first opening and a spaced apart second opening therein with a fluid path in the cavity between the first and second openings. The first opening has a first coupler that is adapted to operably connect to an endotracheal tube. The second opening has a second coupler that is adapted to operably connect to a PEEP valve. The apparatus may include a third opening in the body. The third opening has a third coupler that is adapted to operably connect to an enhanced oxygen source. The third opening in the body is operably located between the first opening and the second opening.

### BACKGROUND OF THE INVENTION

The present disclosure relates to PEEP valves and endotracheal tubes. This may, in one example, include an adaptor for use with an endotracheal tube and a PEEP Valve. During medical treatment of patients requiring breathing assistance, it is frequently necessary to insert an endotracheal tube into the trachea of the patient. Often, it would be desirable to include a Positive End Expiratory Pressure Valve and an enhanced oxygen source within a system including an endotracheal tube. Thus, there is a need for improvement in this field.

### SUMMARY OF THE INVENTION

In one exemplary embodiment, an apparatus for use with an endotracheal tube is disclosed. The apparatus may include a body defining a cavity therein. The body may have a first opening and a spaced apart second opening therein with a fluid path in the cavity between the first and second openings. The first opening may have a first coupler that is adapted to operably connect to an endotracheal tube. The second opening may have a second coupler that is adapted to operably connect to a PEEP valve. The apparatus may further include a third opening in the body. The third opening may have a third coupler that is adapted to operably connect to an enhanced oxygen source.

The third opening in the body may be located along said fluid path between the first opening and the second opening. The third opening may include a ribbed oxygen port adapted to operably connect to an enhanced oxygen source.

The body may be made of plastic. The body may be transparent.

The first opening may be circular having a first diameter. The second opening may be circular having a second diameter that is greater than the first diameter.

Further, the body may have a frustoconical portion that tapers from the first opening to said second opening.

The first coupler may include a generally cylindrical portion of an inner surface of the body with a slight taper. The inner surface may operably connect to a connector of an endotracheal tube using a friction fit between the first coupler and the connector.

The second coupler may be a generally cylindrical portion of an outer surface of the body with a slight taper. The outer surface may operably connect to a connecting portion of a PEEP valve using a friction fit between the second coupler and the connecting portion.

Further disclosed is a kit comprising the apparatus as described above sterilized and contained within a sterile package. The kit may further include a sterilized endotracheal tube. The kit may further include a sterilized PEEP valve.

Further disclosed is an apparatus for use with an endotracheal tube. The apparatus may include a PEEP valve having a body optionally extending therefrom and defining a cavity therein. The body may have a first opening. The first opening may have a first coupler that is adapted to operably connect to an endotracheal tube to form a fluid path between the PEEP valve and the endotracheal tube. The apparatus may further include a second opening in the body that may be fluidly connected to the fluid path. The second opening may have a second coupling element that may be adapted to operably connect to an enhanced oxygen source.

The second opening in the body may be located along the fluid path between the first opening and the PEEP valve. The second opening may include a ribbed oxygen port adapted to operably connect to an enhanced oxygen source.

The first opening may be circular having a first diameter. The second opening may be circular having a second diameter that is smaller than the first diameter.

The first coupler may include a generally cylindrical portion of an inner surface of the body with a slight taper. The inner surface may operably connect to a connector of an endotracheal tube using a friction fit between the first coupler and the connector.

Further disclosed is a kit comprising the apparatus as described above sterilized and contained within a sterile package. The kit may further include a sterilized endotracheal tube.

In additional examples, an apparatus for use with an endotracheal tube is disclosed. The apparatus may include an endotracheal tube having a body extending therefrom and defining a cavity therein. The body may have a first opening. The first opening may have a first coupler that may be adapted to operably connect to a PEEP valve to form a fluid path between the PEEP valve and the endotracheal tube. The apparatus may further include a second opening in the body that is fluidly connected to the fluid path. The second opening may have a second coupling element that may be adapted to operably connect to an enhanced oxygen source.

The second opening in the body may be located along the fluid path between the first opening and the endotracheal tube. The second opening may include a ribbed oxygen port adapted to operably connect to an enhanced oxygen source.

The first opening may be circular having a first diameter. The second opening may be circular having a second diameter that is smaller than said first diameter.

The first coupler may be a generally cylindrical portion of an outer surface of the body with a slight taper. The outer surface may operably connect to a connecting portion of a PEEP valve using a friction fit between the second coupler and the connecting portion.

Further disclosed is a kit comprising the apparatus as described above sterilized and contained within a sterile package. The kit may further include a sterilized PEEP valve.

### BRIEF DESCRIPTION OF THE DRAWINGS

FIG. 1 illustrates a system including an adaptor operably connected to an endotracheal tube, PEEP Valve and oxygen line.

FIG. 2 is an exploded view of the system of FIG. 1.

FIG. 3 is a perspective exploded view of the system of FIG. 1.

FIG. 4 is a perspective view of the adaptor.

FIG. 5 is another perspective view of the adaptor.

FIG. 6 is a side view of the adaptor.

FIG. 7 is a top view of the adaptor.

FIG. 8 is a bottom view of the adaptor.

FIG. 9 is a cross-sectional side view of the adaptor.

FIG. 10 illustrates the adaptor in a kit.

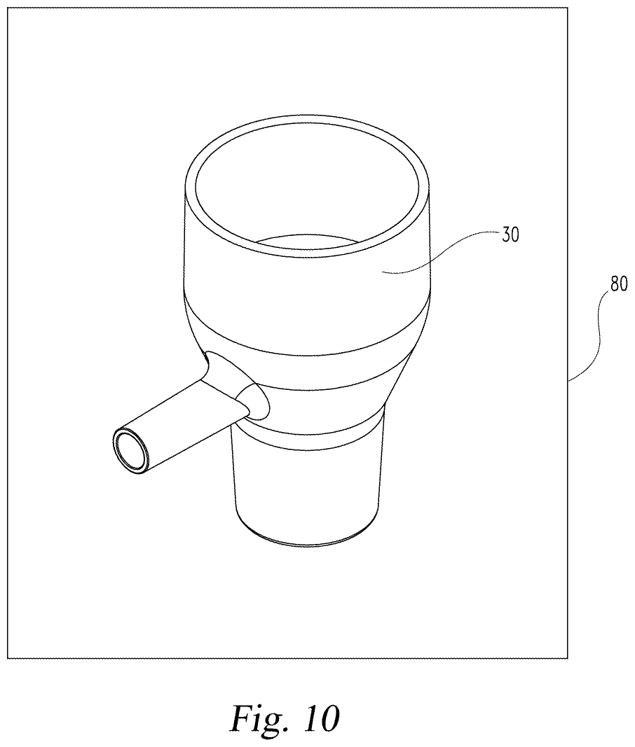

FIG. 11 illustrates is a schematic diagram view of a PEEP valve connected to an endotracheal tube.

### DETAILED DESCRIPTION OF THE INVENTION

For the purpose of promoting an understanding of the principles of the disclosure, reference will now be made to the embodiments illustrated in the drawings and specific language will be used to describe the same. It will nevertheless be understood that no limitation of the scope of the disclosure is thereby intended. Any alterations and further modifications in the described embodiments, and any further applications of the principles of the disclosure as described herein are contemplated as would normally occur to one skilled in the art to which the disclosure relates. One embodiment of the disclosure is shown in great detail, although it will be apparent to those skilled in the relevant art that some features that are not relevant to the present disclosure may not be shown for the sake of clarity.

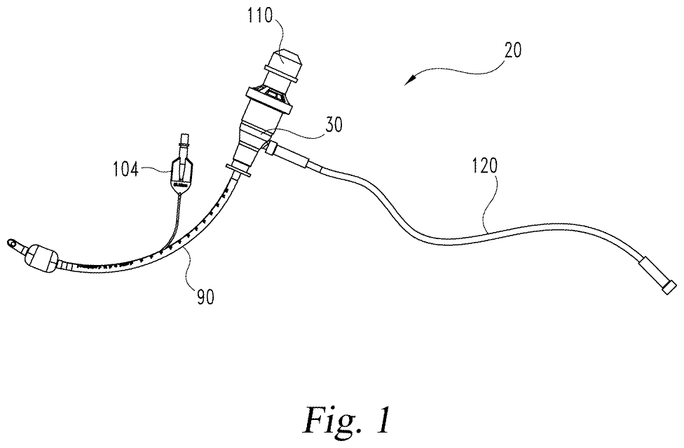

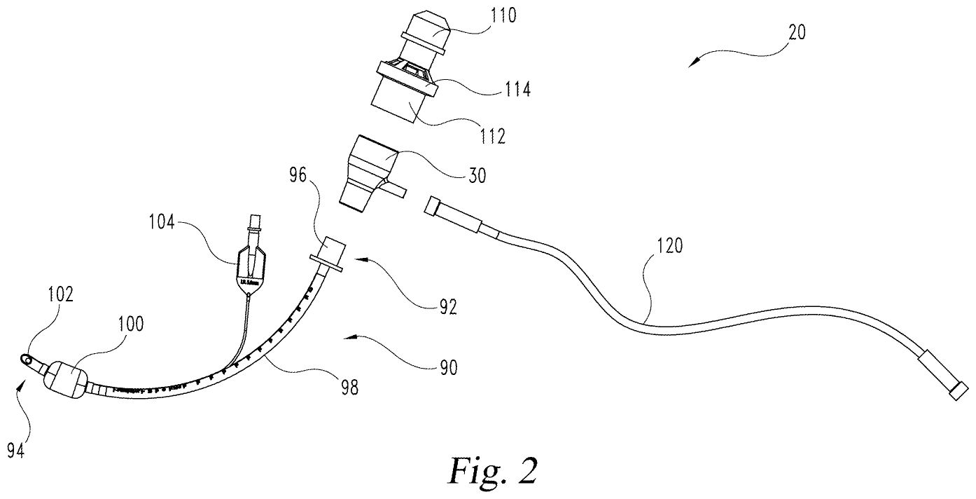

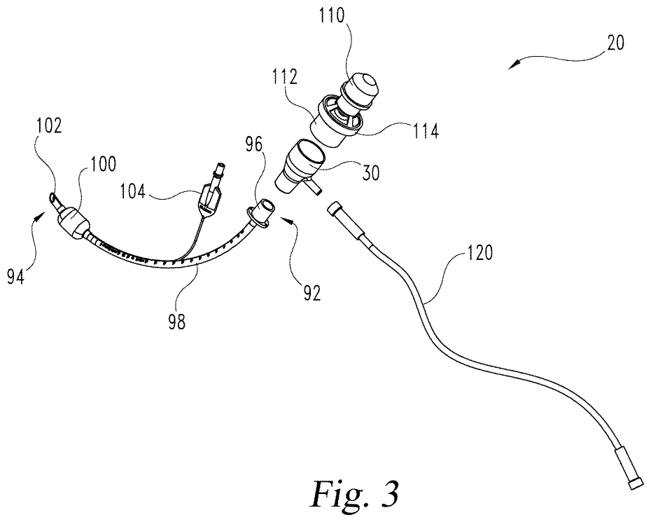

As illustrated in FIG. 1, the present disclosure involves a system 20 including an adaptor 30 that may be used with an endotracheal tube 90 and a PEEP valve 110. FIG. 1 illustrates the system 20 as coupled together. FIGS. 2 and 3 illustrate the system 20 in partially exploded views. The endotracheal tube 90 may include a proximal end 92 and a distal end 94. The proximal end 92 may include a coupler/connector 96 capable of operably coupling to the adaptor. A tubing 98 may extend from the proximal end 92 to the distal end 94. The distal end 94 may include a cuff 100 and a beveled tip 102. Often, the bevel 102 may include a Murphy's Eye (not show). The endotracheal tube 90 may include a Magill Curve. The endotracheal tube 90 may further include a pilot balloon 104.

The PEEP valve 110 may include a coupler 112 configured to operably couple to the adaptor 30. The PEEP valve 110 may further include a biological filter integrated within a housing 114 of the PEEP valve 110. The biological filter prevents particles from the patient's exhaled breath from entering the atmospheric air.

Further, the system 20 may include a gas connector 120 to connect to a source of gas, for example a supplemental oxygen source (not shown). The gas connector may be any of the well-known structures used to connect to a gas source and/or compressed breathable gas.

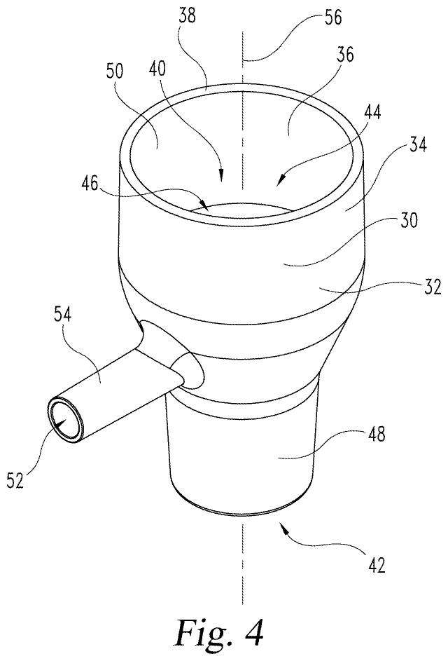

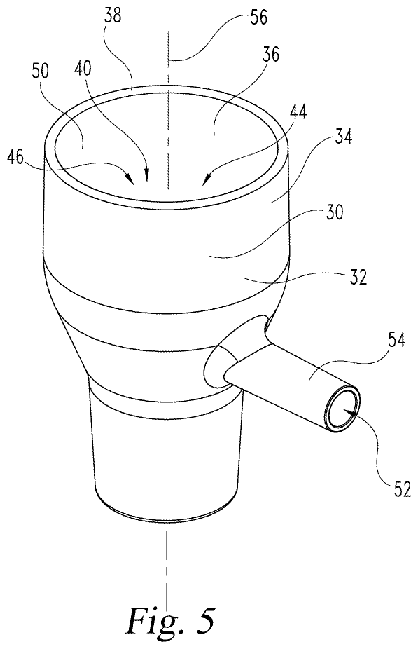

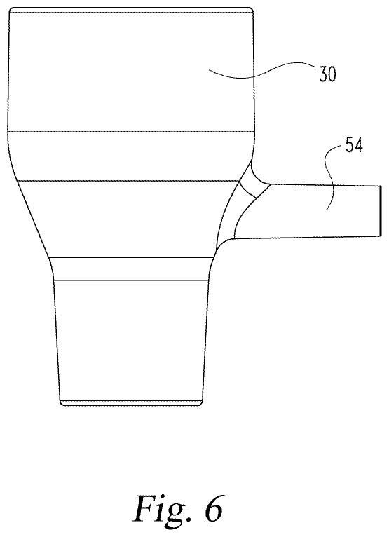

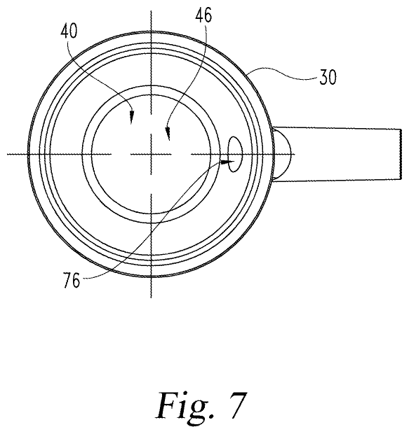

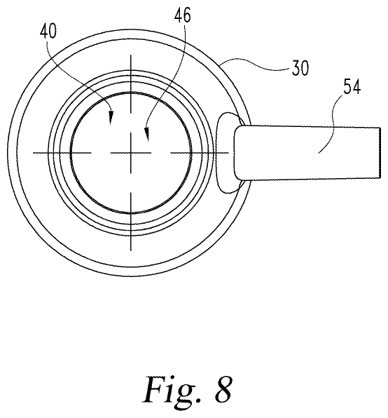

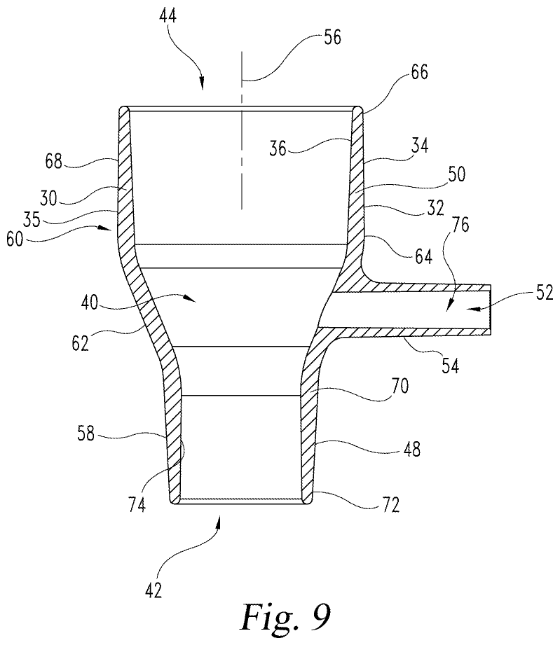

Turning to FIGS. 4-9, the adaptor 30 is illustrated without the rest of the system. The adaptor 30 may include a body 32 with an outer surface 34 and an inner surface 36. The outer surface 34 and inner surface 36 may include a thickness 38 between the two surfaces. The body 32 may define a cavity 40 therein. The body 32 may include a first opening 42 and a spaced apart second opening 44 with a fluid path 46 in said cavity 40 between said first and second openings 42 and 44. The first opening 42 may include a first coupler 48 that is adapted to operably connect to an endotracheal tube. The second opening 44 may include a second coupler 50 that is adapted to operably connect to a PEEP valve. In some embodiments, the adaptor 30 optionally includes a third opening 52 in the body 32. The third opening 52 may include a third coupler 54 that is adapted to operably connect to an enhanced oxygen source. In some examples, the third opening 52 in the body 32 is operably located between the first opening 42 and the second opening 44. The third coupler 54 may include an optionally ribbed (circumferentially) oxygen port adapted to operably connect to an enhanced oxygen source. Optionally, coupler 54 and/or gas connector 120 may include a spigot or other valve (not shown) to open, close and/or restrict gas flow.

The first opening 42 may be circular having a first diameter. In other embodiments, the first opening 42 may be any appropriate shape. In some examples, first opening 42 is a female conical connector. In some examples, the first diameter may be about 15 mm. The female conical connector may be compliant with BS EN ISO 5356-1 or other equivalent standards. The second opening 44 may also be circular having a second diameter that is greater than said first diameter. In other embodiments, the second opening 44 may be any appropriate shape. In some examples, the second opening is a male conical connector. In some examples, the second diameter may be about 30 mm. The male conical connector may be compliant with BS EN ISO 5356-1 or other equivalent standards. Those dimensions are not limiting unless expressly, numerically claimed. It is understood that in some embodiments the first opening 42 may be a male conical connector and the second opening may be a female conical connector.

The body 32 may have a frustoconical portion that tapers from first opening 42 to second opening 44. The third opening 52 may be circular having a third diameter. In other embodiments, the third opening 52 may be any appropriate shape. In some examples, the third opening 52 may be a male connector. In some examples, third diameter may have an outer diameter of about 3.175-9.525 mm. In some preferred embodiments the outer diameter is about 6.35 mm. The third diameter may have an inner diameter of about 1.7-6.35 mm. In some preferred embodiments the inner diameter is about 3.175 mm. Those dimensions are not limiting unless expressly, numerically claimed. In other embodiments, the third opening may be a female connector.

In some embodiments, the body 32 may be made of any suitable material, for example plastic. In other embodiments, the body may be transparent. Optionally, body 32 and coupler 54 are molded as a unitary piece. Optionally, in addition to coupler 54, other couplers (not shown) to body 32 may be provided affording fourth, fifth or other amounts of openings (not shown). These may, optionally, provide the ability to introduce moisture and/or medicaments into the air flow.

Body 32 may include a central longitudinal axis 56. The body 32 may include a cylindrical surface/wall 35. The body 32 may include a first section 58 and a second section 60. The first section 58 of the cavity may include a first volume. The second section 60 of the cavity includes a second volume. In some examples, the first volume is smaller relative to the volume of the second volume. In some embodiments the body 32 may include a tapered section 62 between and connecting the first section 58 of the body 32 and the second section 60 of the body 32.

The first section 58 of the body 32 may include the first coupler 48. The first coupler 48 may include a first portion 70 and a second portion 72. The wall 35 of the body 32 is thicker at the first portion 70 of the first coupler 48 than the second portion 72 of the first coupler 48. An inner surface 36 includes a tapered surface 74 tapering outward away from the central longitudinal axis 56 along at least a portion of the distance between the first portion 70 to the second portion 72 of the first coupler 48. In some examples, the tapered surface 74 tapering along the entire distance between the first portion 70 to the second portion 72 of the first coupler 48. The connector 96 of the endotracheal tube 90 is inserted into the first coupler 48 so that an outer surface of the connector 96 of the endotracheal tube 90 is connected to the tapered surface 74 of the first coupler 48 through a friction fit.

The second section 60 of the body 32 may include the second coupler 50. The second coupler 50 may include a first portion 64 and a second portion 66. The wall 35 of the body 32 is thicker at the first portion 64 of the second coupler 50 than the second portion 66 of the second coupler 50. An outer surface 34 includes a tapered surface 68 that tapers inward toward the central longitudinal axis 56 along at least a portion of the distance between the first portion 64 to the second portion 66 of the second coupler 50. In some examples, the tapered surface 68 tapers along the entire distance between the first portion 64 to the second portion 66 of the second coupler 50. The PEEP Valve 110 is inserted over the second coupler 50 so that an inner surface of the coupler 112 of the PEEP valve 110 is connected to the tapered surface 68 of the second coupler 50 through a friction fit.

In some embodiments, an outer surface of the first section operably connects to an inner surface of the connector of the endotracheal tube through a friction fit, similar to the connection discussed in detail regarding the second section and the PEEP valve above. In the same or a different embodiment, an inner surface of the second section operably connects to an outer surface of the coupler of the PEEP valve through a friction fit, similar to the connection discussed in detail regarding the first section and the endotracheal tube above.

In some embodiments, the first coupler and second coupler are threaded to match threaded portions on the connector of the endotracheal tube and the coupler of the PEEP valve, respectively. In other embodiments, the couplers may include a tooth or set of teeth. In this embodiment, the PEEP valve or connector of the endotracheal tube may include teeth that correspond with the teeth of the couplers. This allows the PEEP valve or the connector of the endotracheal tube to be extended over or within the coupler and then turned and locked into place. Any reasonable connection method may be used.

The body 32 may further define a second fluid path 76 through from the third opening 52 that fluidly connects to the first fluid path 46. The second fluid path 76 may flow perpendicular to the central longitudinal axis 56 of the body 32.

The components of system may be surgically sterilized and contained within a sterile package/kit 80. The components (discussed above) may all come in one kit or may be sold separately. For example, one kit may include all of the components discussed above. In other examples, the adaptor may be packaged and sold in a kit as an individual component.

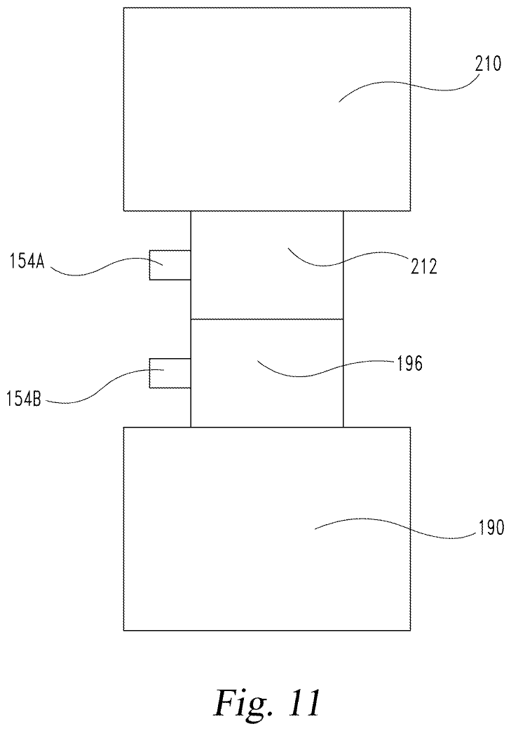

Turning to FIG. 11, a schematic diagram view of a PEEP valve 210 connected to an endotracheal tube 190 is shown. In one embodiment, a coupler 212 of PEEP valve 210 includes the characteristics of adaptor 30 integrated into the coupler 212, such that the coupler 212 may be adapted to directly connect to an endotracheal tube 190. Therefore, the applicable discussion regarding adaptor 30 is incorporated herein. In some embodiments, coupler 212 operably connects to connector/coupler 196 of the endotracheal tube 190.

Further, a second coupler 154A may be adapted to operably connect to an enhanced oxygen source. In some examples, the second coupler 154A may be operably located between the coupler 212 and the PEEP valve 210. The second coupler 154A may include a ribbed oxygen port adapted to operably connect to an enhanced oxygen source.

In another embodiment, a connector 196 of endotracheal tube 190 includes the characteristics of adaptor 30 integrated into the connector 196, such that the connector 196 may be adapted to directly connect to a PEEP valve 210. Therefore, the applicable discussion regarding adaptor 30 is incorporated herein. In some embodiments, connector 196 operably connects to coupler 212 of the PEEP valve 210.

Further, a second coupler 154B may be adapted to operably connect to an enhanced oxygen source. In some examples, the second coupler 154B may be operably located between the connector 196 and the endotracheal tube 190. The second coupler 154B may include a ribbed oxygen port adapted to operably connect to an enhanced oxygen source.

### Glossary of Terms

With respect to the specification and claims, it should be noted that the singular forms “a”, “an”, “the”, and the like include plural referents unless expressly discussed otherwise. As an illustration, references to “a device” or “the device” include one or more of such devices and equivalents thereof. It also should be noted that directional terms, such as “up”, “down”, “top”, “bottom”, and the like, are used herein solely for the convenience of the reader in order to aid in the reader's understanding of the illustrated embodiments, and it is not the intent that the use of these directional terms in any manner limit the described, illustrated, and/or claimed features to a specific direction and/or orientation.

While the disclosure has been illustrated and described in detail in the drawings and foregoing description, the same is to be considered as illustrative and not restrictive in character, it being understood that only the preferred embodiment has been shown and described and that all changes, equivalents, and modifications that come within the spirit of the inventions defined by following claims are desired to be protected. All publications, patents, and patent applications cited in this specification are herein incorporated by reference as if each individual publication, patent, or patent application were specifically and individually indicated to be incorporated by reference and set forth in its entirety herein.

The language used in the disclosure are presumed to have only their plain and ordinary meaning, except as explicitly defined below. The words used in the definitions included herein are to only have their plain and ordinary meaning. Such plain and ordinary meaning is inclusive of all consistent dictionary definitions from the most recently published Webster's and Random House dictionaries. As used herein, the following definitions apply to the following terms or to common variations thereof (e.g., singular/plural forms, past/present tenses, etc.):

“About” with reference to numerical values generally refers to plus or minus 10% of the stated value. For example, if the stated value is 4.375, then use of the term “about 4.375” generally means a range between 3.9375 and 4.8125.

“Ambient” is the surrounding atmosphere and its conditions. When a concrete pressure is needed for measurement and/or comparison purposes, it is measured at or relative to sea level.

“And/or” is inclusive here, meaning “and” as well as “or”. For example, “P and/or Q” encompasses, P, Q, and P with Q; and, such “P and/or Q” may include other elements as well.

“Biological filter” generally refers to the filters known in the art that trap particles exhaled by the patient and keeps those particles from exiting into the atmosphere, including those used in face masks or filters traps.

“Cavity” as used herein generally refers to an empty space in a solid object. The cavity can be completely or partially surrounded by the solid object. For example, the cavity can be open to the surrounding environment.

“Compressed breathable gas” is a source of compressed gas, for example oxygen, or air and oxygen, or nitrogen and oxygen, or medical gas, or otherwise breathable by humans and pressurized above ambient. Sources of compressed gas may include that supplied by air pumps, pressurized medical gas tanks, oxygen concentrators or portable air compressors.

“Coupler” as used herein means a thing that connects two things, especially mechanical components or systems.

“Fluid” as used herein means any liquid or gas.

“multiple” as used herein is synonymous with the term “plurality” and refers to more than one, or by extension, two or more.

“Optionally” as used herein means discretionary; not required; possible, but not compulsory; left to personal choice.

“Oxygen source” as used herein the source of the oxygen (normally enhanced from ambient) being provided into the gas flow path of the flow generator. For example, atmospheric air or oxygen from an artificial source, such as an oxygen tank.

“Path” as used herein means the route of flow of air from the starting point through the resuscitation system to the patient.

“Positive End Expiratory Pressure Valve or PEEP Valve” as used herein means a valve generally used to maintain a predetermined pressure level in the lungs of a patient who is being ventilated with oxygen or air. Typical PEEP valves include a spring biased relief valve which remains closed and prevents the patient from exhaling until the pressure of the patient's exhalation gas exceeds the force of the spring after which the valve opens and the patient's exhalation gas is exhausted through an exit port on the PEEP valve and into the atmosphere.

“Substantially” generally refers to the degree by which a quantitative representation may vary from a stated reference without resulting in an essential change of the basic function of the subject matter at issue. The term “substantially” is utilized herein to represent the inherent degree of uncertainty that may be attributed to any quantitative comparison, value, measurement, and/or other representation.

1. An apparatus for use with an endotracheal tube comprising:
a body defining a cavity therein; and
wherein the body has a first opening and a spaced apart second opening therein with a fluid path in said cavity between said first and second openings;
wherein the first opening has a first coupler that is adapted to operably connect to an endotracheal tube;
wherein the second opening has a second coupler that is adapted to operably connect to a PEEP valve; and
a third opening in said body, wherein the third opening has a third coupler that is adapted to operably connect to an enhanced oxygen source.

2. The apparatus of claim 1, wherein the third opening in said body is located along said fluid path between the first opening and the second opening.

3. The apparatus of claim 1, wherein the third opening includes a ribbed oxygen port adapted to operably connect to an enhanced oxygen source.

4. The apparatus of claim 1, wherein the body is made of plastic.

5. The apparatus of claim 1, wherein the body is transparent.

6. The apparatus of claim 1, wherein said first opening is circular having a first diameter, wherein said second opening is circular having a second diameter that is greater than said first diameter.

7. The apparatus of claim 1, wherein said body has a frustoconical portion that tapers from said first opening to said second opening.

8. The apparatus of claim 1, wherein the first coupler includes a generally cylindrical portion of an inner surface of the body with a slight taper, wherein the inner surface operably connects to a connector of an endotracheal tube using a friction fit between the first coupler and the connector.

9. The apparatus of claim 1, wherein the second coupler is a generally cylindrical portion of an outer surface of the body with a slight taper, wherein the outer surface operably connects to a connecting portion of a PEEP valve using a friction fit between the second coupler and the connecting portion.

10. A kit comprising the apparatus of claim 1 sterilized and contained within a sterile package.

11. The kit of claim 10 and further comprising a sterilized endotracheal tube.

12. The kit of claim 11 and further comprising a sterilized PEEP valve.

13. An apparatus for use with an endotracheal tube comprising:
a PEEP valve having a body extending therefrom and defining a cavity therein; and
wherein the body has a first opening, wherein the first opening has a first coupler that is adapted to operably connect to an endotracheal tube to form a fluid path between said PEEP valve and said endotracheal tube; and
a second opening in the body that is fluidly connected to the fluid path, wherein the second opening has a second coupling element that is adapted to operably connect to an enhanced oxygen source.

14. The apparatus of claim 13, wherein the second opening in said body is located along said fluid path between the first opening and the PEEP valve.

15. The apparatus of claim 13, wherein the second opening includes a ribbed oxygen port adapted to operably connect to an enhanced oxygen source.

16. The apparatus of claim 13, wherein said first opening is circular having a first diameter, wherein said second opening is circular having a second diameter that is smaller than said first diameter.

17. The apparatus of claim 13, wherein the first coupler includes a generally cylindrical portion of an inner surface of the body with a slight taper, wherein the inner surface operably connects to a connector of an endotracheal tube using a friction fit between the first coupler and the connector.

18. A kit comprising the apparatus of claim 13 sterilized and contained within a sterile package.

19. The kit of claim 18 and further comprising a sterilized endotracheal tube.

20. An apparatus for use with an endotracheal tube comprising:
an endotracheal tube having a body extending therefrom and defining a cavity therein; and
wherein the body has a first opening, wherein the first opening has a first coupler that is adapted to operably connect to a PEEP valve to form a fluid path between said PEEP valve and said endotracheal tube; and
a second opening in the body that is fluidly connected to the fluid path, wherein the second opening has a second coupling element that is adapted to operably connect to an enhanced oxygen source.

21. The apparatus of claim 20, wherein the second opening in said body is located along said fluid path between the first opening and the endotracheal tube.

22. The apparatus of claim 20, wherein the second opening includes a ribbed oxygen port adapted to operably connect to an enhanced oxygen source.

23. The apparatus of claim 20, wherein said first opening is circular having a first diameter, wherein said second opening is circular having a second diameter that is smaller than said first diameter.

24. The apparatus of claim 20, wherein the first coupler is a generally cylindrical portion of an outer surface of the body with a slight taper, wherein the outer surface operably connects to a connecting portion of a PEEP valve using a friction fit between the first coupler and the connecting portion.

25. A kit comprising the apparatus of claim 20 sterilized and contained within a sterile package.

26. The kit of claim 25 and further comprising a sterilized PEEP valve.
